(19) United States
(12) Patent Application Publication
(10) Pub. No.: US 2021/0407621 A1
(43) Pub. Date: Dec. 30, 2021

(54) PRENATAL PURITY ASSESSMENTS USING BAMBAM

(71) Applicant: NantOmics, LLC, Culver City, CA (US)

(72) Inventors: John Zachary Sanborn, Culver City, CA (US); Mark Johnson, Culver City, CA (US)

(21) Appl. No.: 17/278,236

(22) PCT Filed: Sep. 20, 2019
(86) PCT No.: PCT/US2019/052218

§ 371 Date: Mar. 19, 2021

Related U.S. Application Data
(60) Provisional application No. 62/745,163, filed on Oct. 12, 2018.

(51) Int. Cl. G16B 20/20; G16B 30/10

(52) U.S. Cl. CPC G16B 20/20; G16B 30/10

(57) ABSTRACT

Contemplated systems and methods are directed to detecting and quantifying purity of a fetal DNA sample with respect to contamination with maternal DNA.

This application claims priority to our copending US provisional patent application with the Ser. No. 62/745,163, which was filed Oct. 12, 2019, and which is incorporated by reference herein.

### FIELD OF THE INVENTION

The field of the invention is omics analysis of fetal DNA, especially as it relates to fetal DNA analysis from maternal blood.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Prenatal diagnosis of an embryo or fetus is commonly performed for a variety of reasons, including identification of gender, detection of genetic abnormalities or genetic predisposition to a disease or disorder, and paternity determination. For example, among other known methods, mass genomic sequencing, allele specific sequencing, or allele specific PCR are described in U.S. Pat. Nos. 7,332,277, 8,442,774, and 8,972,202. While conceptually relatively simple, some of these methods are confounded by contamination of the fetal nucleic acid with nucleic acids from the maternal side. Resolution of maternal and fetal DNA has been attempted by analysis of multiple polymorphic sites as is described in WO2013/130848. However, such analysis of often time consuming and requires a priori knowledge of target sites.

### SUMMARY OF THE INVENTION

The inventive subject matter is directed to various systems, computer readable media, and computer implemented methods of identifying purity of a fetal DNA with respect to contamination by maternal DNA.

Most preferably, contemplated methods will include a step of preparing or obtaining sequencing data obtained from a sample comprising fetal DNA, and sequencing data obtained from a sample comprising maternal DNA, a step of comparing the sequencing data obtained from the sample comprising fetal DNA with the sequencing data obtained from the sample comprising maternal DNA to thereby detect variants; a step of calculating a difference in allele fractions using the variants of the fetal DNA and the variants of the maternal DNA, and a further step of calculating purity using a distribution of difference in allele fractions.

In further preferred aspects, the sample comprising the fetal DNA will comprise or be a fraction of whole blood. Most typically, but not necessarily, the sequencing data are whole genome sequencing data, and/or the step of comparing comprises an incremental location-guided alignment. In further contemplated aspects, the step of calculating will include identifying a peak value in the distribution of difference in allele fractions and multiplying the peak value by 2. Moreover, it is contemplated that the step of calculating the difference in allele fraction may include a step of determination of allele fractions AF

$${AF}_{M + D} = \frac{{M}_{B} ( 1 - α ) + {D}_{B} α}{( {M}_{A} + {M}_{B} ) ( 1 - α ) + ( {D}_{A} + {D}_{B} ) α}$$

wherein M_{A} and M_{B} or D_{A} and D_{B} are the copy numbers of the A and B alleles in the maternal (or daughter) sample, respectively, and wherein M_{A}+M_{B}=2 or D_{A} and D_{B}=2 for a diploid genome, and the step of calculating the difference in allele fraction may be determined using

$$Δ AF = {AF}_{M + D} - {AF}_{M} = \frac{{M}_{B} ( 1 - α ) + {D}_{B} α}{( {M}_{A} + {M}_{B} ) ( 1 - α ) + ( {D}_{A} + D ) α} - \frac{{M}_{B}}{( {M}_{A} + {M}_{B} )} .$$

Additionally, it is contemplated that the step of calculating the purity is determined using

$$α = \vert \frac{2 Δ AF}{{F}_{B} - {M}_{B}} \vert .$$

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary CEPH pedigree.

FIG. 2 depicts an exemplary true (simulated) purity of 10%, with an estimated purity of 9% according to the inventive subject matter.

FIG. 3 depicts an exemplary true (simulated) purity of 50%, with an estimated purity of 47% according to the inventive subject matter.

FIG. 4 depicts an exemplary true (simulated) purity of 100%, with an estimated purity of 100%.

FIG. 5 depicts an exemplary summary of results correlating true (simulated) purity versus estimated purity according to the inventive subject matter.

### DETAILED DESCRIPTION

The inventors have now discovered that contamination of fetal DNA with maternal DNA can be identified and resolved using a process in which samples enriched in maternal and fetal DNA are compared, preferably in a synchronous incremental process to so allow for a method to estimate purity of prenatal samples extracted from the mother. To that end, the inventors used the sequencing data from cells of known pedigree (e.g., origin and familial relationship), which were used as test samples in computational systems and methods as are described in more detail below.

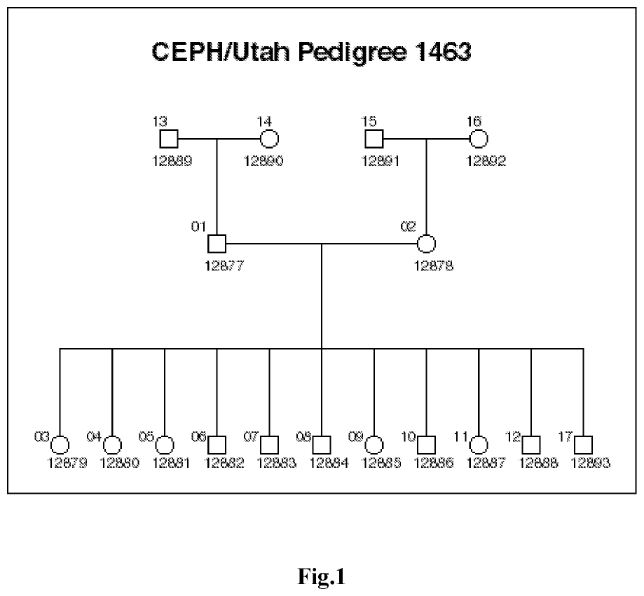

To estimate the purity from in-silico mixtures of Maternal+Daughter cell lines, the inventors used whole exome sequencing data from two cell lines derived from the CEPH/Utah family pedigree 1463: GM12878 (mother, M) and GM12887 (daughter, D), and an the CEPH pedigree is shown in FIG. 1. Each sample was sequenced in two replicates, where each replicate meets or exceeds an average exome coverage of 250×.

Using an in-silico mixing approach, 9 mixtures of the raw sequencing data for GM12878 (M) and GM12887 (D) were generated to model the following “true” (or simulated) purity percentages: 5%, 7.5%, 10%, 15%, 20%, 30%, 40%, 50%, and 100%. Each mixture was generated by sampling paired sequencing reads from a single replicate of each source dataset at a rate according to the desired purity, a, (where 0≤α≤1). This can be performed using a Monte Carlo method to select reads from both source datasets, where the probability of sampling a read pair from the Mother (M) and Daughter (D) sequencing datasets is as follows:

Pr(Sampling Read Pair from M|α)=(1−α)

Pr(Sampling Read Pair from D|α)=α

The sequencing data for each mixture are aligned using an incremental location-guided alignment, and most preferably the NantOmics alignment pipeline (or other aligner that preferably generates a SAM, BAM, or GAR file) to generate a single BAM file for each mixture and replicate. Each mixture (M+D) is then compared to the aligned sequencing data from GM12878 (M) by the NantOmics variant processing pipeline (BAMBAM, see e.g., U.S. Pat. No. 9,824,181). This process utilizes a substantially identical approach to the GPS tumor vs. matched normal processing, where the M sequence is treated as a “matched-normal” and the D sequence is treated as a “tumor”. The process generates both “somatic” and “germline” variant calls, where in this case “somatic” calls are those inherited from the father (GM12877) and “germline” calls are those inherited from the mother. Note that a small percentage of “somatic” calls may be de novo variants acquired somatically (i.e. not inherited from either parent) in the D genome, but the de novo contribution can be treated as paternal variants for the purposes of the analysis below. Furthermore, it should be noted that variants classified as “germline” may also be inherited from the father wherever both mother and father share the same genetic variant.

The allele fractions (AF) of both somatic and germline variants are calculated in both the M+D mixture and M sequencing datasets for all common single nucleotide variants (population allele frequency >5%) that have total read depth >50 in both M+D and M. Table 1 below lists the number of variants (SNV counts) identified in each mixture:

| True Purity | Replicate | # “Somatic” | # “Germline” |
|---|---|---|---|
| 5 | 1 | 4,732 | 38,018 |
| | 2 | 4,512 | 38,142 |
| 7.5 | 1 | 4,742 | 37,928 |
| | 2 | 4,472 | 37,459 |
| 10 | 1 | 4,653 | 36,123 |
| | 2 | 4,469 | 37,289 |
| 15 | 1 | 6,704 | 38,699 |
| | 2 | 6,662 | 39,185 |
| 20 | 1 | 6,540 | 37,126 |
| | 2 | 6,561 | 37,800 |
| 30 | 1 | 6,901 | 38,160 |
| | 2 | 7,053 | 39,175 |
| 40 | 1 | 6,993 | 38,425 |
| | 2 | 6,840 | 37,723 |
| 50 | 1 | 7,124 | 39,173 |
| | 2 | 7,017 | 38,232 |
| 100 | 1 | 6,767 | 31,621 |
| | 2 | 6,532 | 30,436 |

To estimate the purity level of the M+D mixture, it should be noted that variants should have the following expected variant allele fractions (AFs), where “A”=reference allele and “B” is the variant allele, given a mixture fraction (α):

$${AF}_{M + D} = \frac{{M}_{B} ( 1 - α ) + {D}_{B} α}{( {M}_{A} + {M}_{B} ) ( 1 - α ) + ( {D}_{A} + {D}_{B} ) α}$$

where M_{A} and M_{B} (or D_{A} and D_{B}) are the copy numbers of the A and B alleles in the maternal (or daughter) sample, respectively, where M_{A}+M_{B}=2 (or D_{A} and D_{B}=2) for a diploid genome.

Delta AF is determined by subtracting the AF from the maternal sample (AF_{M}) from that of the mixture sample (AF_{M+D}):

$$Δ AF = {AF}_{M + D} - {AF}_{M} = \frac{{M}_{B} ( 1 - α ) + {D}_{B} α}{( {M}_{A} + {M}_{B} ) ( 1 - α ) + ( {D}_{A} + D ) α} - \frac{{M}_{B}}{( {M}_{A} + {M}_{B} )}$$

which simplifies to:

ΔAF=½(D_{E}−M_{E})α.

Alternatively, solving for α, and taking the absolute value, one can estimate purity as:

$$α = \vert \frac{2 Δ AF}{{F}_{B} - {M}_{B}} \vert$$

One can then determine what D_{B} and M_{B} should be for all likely Mendelian combinations from an assumed paternal contribution:

Maternal AA+Daughter AB (Paternal Contribution=B):

α(M_{B}=0,D_{B}=1)=2|ΔAF|

Maternal AB+Daughter AA (Paternal Contribution=A):

α=(M_{B}=1,D_{B}=0)=2|ΔAF|

Maternal BB+Daughter AB (Paternal Contribution=A):

α=(M_{B}=2,D_{B}=1)=2|ΔAF|

Maternal AB+Daughter AB (Paternal Contribution=A or B):

α=(M_{B}=1,D_{B}=1)=Invalid

Maternal BB+Daughter BB (Paternal Contribution=B):

α=(M_{B}=2,D_{B}=2)=Invalid

Note that the equation for a is invalid for the cases where both maternal and daughter genomes are either both heterozygous or both homozygous for the same variant allele, since the equation results in a division by zero. However, since these cases exhibit no change in Delta AF (ΔAF=0) they can be ignored in the analysis that follows.

To estimate a from the data, one first computes ΔAF for all variants detected in each mixture (both somatic and germline) to form a distribution of ΔAF. Note that all AF estimates (AF_{M+D} and AF_{M}) are expected to be noisy due to random sampling errors. However, the peak of this distribution should still approximately relate to purity as the equation, α=2|ΔAF|, suggests. To find the peak, one can utilize a standard peak-calling algorithm on the ΔAF distribution for each mixture and then simply multiply this peak by 2 to determine the sample's purity a.

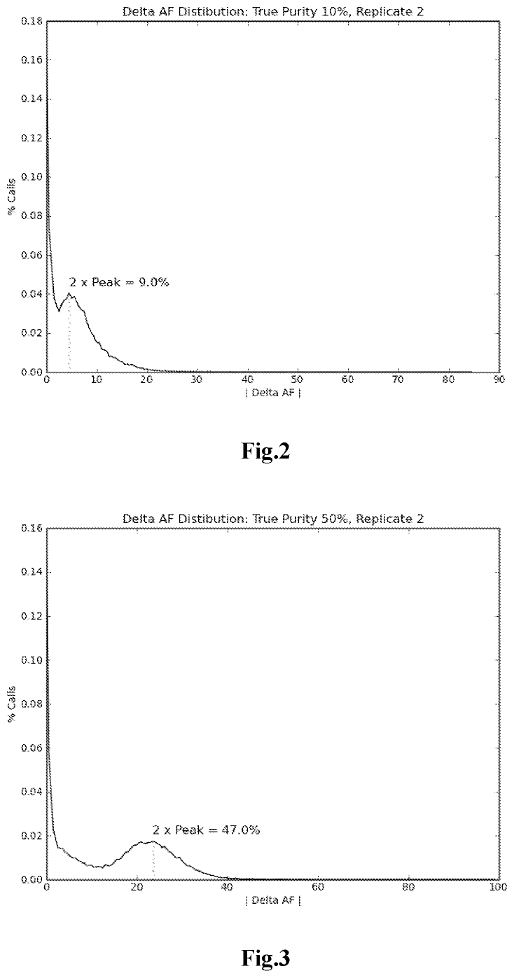

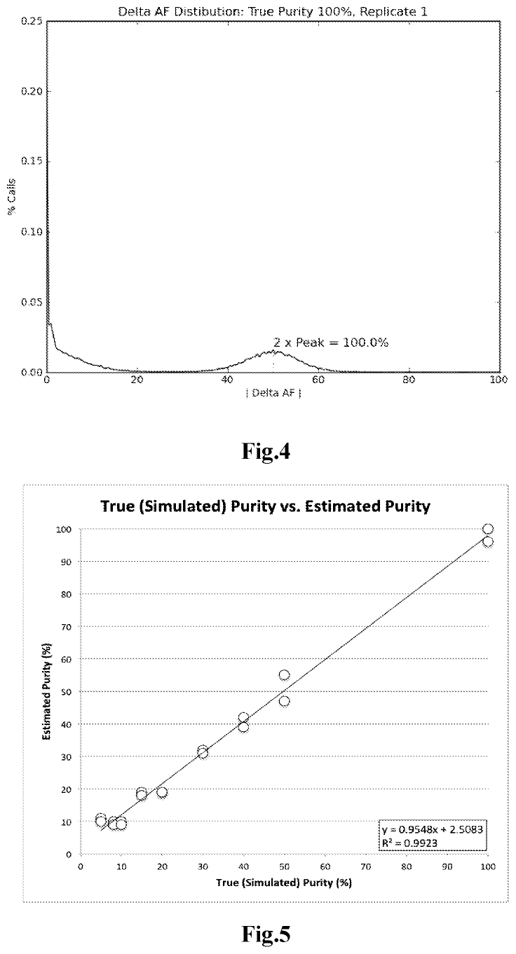

Following the above and in silico mixtures as noted earlier, example plots for the distributions of ΔAF and their estimated purities are shown below for the true (simulated) purities of 10%, 50%, and 100% in FIGS. 2-4 respectively. FIG. 2 shows true (simulated) purity of 10%, with an estimated purity of 9%, FIG. 3 shows true (simulated) purity of 50%, with an estimated purity of 47%, and FIG. 4 shows true (simulated) purity of 100%, with an estimated purity of 100%. This process was repeated for all mixtures noted in Table 1 and replicated, with the results summarized in FIG. 5. As can be taken from the linear regression, the estimated purities track very well with the true purities across a wide range of simulated purities. Further aspects, systems, and methods suitable for use herein are contemplated in our copending International patent application with the serial number PCT/US19/35786, which was filed Jun. 6, 2019, and which is incorporated by reference herein.

It should be noted that any language directed to a computer should be read to include any suitable combination of computing devices, including servers, interfaces, systems, databases, agents, peers, engines, controllers, modules, cloud system, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or otherwise program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, cloud systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

As used in the description herein and throughout the claims that follow, when a system, engine, server, device, module, or other computing element is described as configured to perform or execute functions on data in a memory, the meaning of “configured to” or “programmed to” is defined structurally as one or more processors or cores of the computing element being programmed or otherwise manipulated or altered by a set of software instructions stored in the memory of the computing element to execute the set of functions or operate on target data or data objects stored in the memory.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . , and N, the text should be interpreted as requiring only one element from the group, not A plus N, or B plus N, etc. Moreover, as used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

What is claimed is:

1. A computer implemented method of identifying purity of a fetal DNA with respect to contamination by maternal DNA, comprising:
preparing or obtaining sequencing data obtained from a sample comprising fetal DNA, and sequencing data obtained from a sample comprising maternal DNA;
comparing the sequencing data obtained from the sample comprising fetal DNA with the sequencing data obtained from the sample comprising maternal DNA to thereby detect variants;
calculating a difference in allele fractions using the variants of the fetal DNA and the variants of the maternal DNA; and
calculating purity using a distribution of difference in allele fractions.

2. The method of claim 1, wherein the sample comprising fetal DNA comprises a fraction of whole blood.

3. The method of any one of claims 1-2, wherein the sequencing data are whole genome sequencing data.

4. The method of any one of claims 1-3, wherein the step of comparing comprises an incremental location-guided alignment.

5. The method of any one of claims 1-4, wherein the step of calculating comprises identifying a peak value in the distribution of difference in allele fractions and multiplying the peak value by 2.

6. The method of any one of claims 1-5, wherein the step of calculating the difference in allele fraction uses a step of determination of allele fractions AF
$${AF}_{M + D} = \frac{{M}_{B} ( 1 - α ) + {D}_{B} α}{( {M}_{A} + {M}_{B} ) ( 1 - α ) + ( {D}_{A} + {D}_{B} ) α}$$
wherein M_{A} and M_{B} or D_{A} and D_{B} are the copy numbers of the A and B alleles in the maternal (or daughter) sample, respectively, and wherein M_{A}+M_{B}=2 or D_{A} and D_{B}=2 for a diploid genome.

7. The method of claim 6, wherein the step of calculating the difference in allele fraction is determined using
$$Δ AF = {AF}_{M + D} - {AF}_{M} = \frac{{M}_{B} ( 1 - α ) + {D}_{B} α}{( {M}_{A} + {M}_{B} ) ( 1 - α ) + ( {D}_{A} + D ) α} - \frac{{M}_{B}}{( {M}_{A} + {M}_{B} )} .$$

8. The method of any one of claims 1-7, wherein the step of calculating the purity is determined using
$$α = \vert \frac{2 Δ AF}{{F}_{B} - {M}_{B}} \vert .$$

9. A computer system for identifying purity of a fetal DNA with respect to contamination by maternal DNA, comprising:
a sequence analysis engine coupled to a sequence database that is configured to store sequencing data obtained from a sample comprising fetal DNA, and sequencing data obtained from a sample comprising maternal DNA;
wherein the sequence analysis engine is informationally programmed to
obtain the sequencing data from the sample comprising fetal DNA, and to obtain the sequencing data obtained from the sample comprising maternal DNA;
compare the sequencing data obtained from the sample comprising fetal DNA with the sequencing data obtained from the sample comprising maternal DNA to thereby detect variants;
calculate a difference in allele fractions using the variants of the fetal DNA and the variants of the maternal DNA; and
calculate a purity using a distribution of difference in allele fractions.

10. The computer system of claim 9, wherein the sample comprising fetal DNA comprises a fraction of whole blood.

11. The computer system of any one of claims 9-10, wherein the sequencing data are whole genome sequencing data.

12. The computer system of any one of claims 9-11, wherein the step of comparing comprises an incremental location-guided alignment.

13. The computer system of any one of claims 9-12, wherein the step of calculating comprises identifying a peak value in the distribution of difference in allele fractions and multiplying the peak value by 2.

14. The computer system of any one of claims 9-13, wherein the step of calculating the difference in allele fraction uses a step of determination of allele fractions AF
$${AF}_{M + D} = \frac{{M}_{B} ( 1 - α ) + {D}_{B} α}{( {M}_{A} + {M}_{B} ) ( 1 - α ) + ( {D}_{A} + {D}_{B} ) α}$$
wherein M_{A} and M_{B} or D_{A} and D_{B} are the copy numbers of the A and B alleles in the maternal (or daughter) sample, respectively, and wherein M_{A}+M_{B}=2 or D_{A} and D_{B}=2 for a diploid genome.

15. The computer system of claim 14, wherein the step of calculating the difference in allele fraction is determined using
$$Δ AF = {AF}_{M + D} - {AF}_{M} = \frac{{M}_{B} ( 1 - α ) + {D}_{B} α}{( {M}_{A} + {M}_{B} ) ( 1 - α ) + ( {D}_{A} + D ) α} - \frac{{M}_{B}}{( {M}_{A} + {M}_{B} )} .$$

16. The computer system of any one of claims 9-15, wherein the step of calculating the purity is determined using
$$α = \vert \frac{2 Δ AF}{{F}_{B} - {M}_{B}} \vert .$$

17. A non-transient computer readable medium containing program instructions for causing a computer to perform a method of identifying purity of a fetal DNA with respect to contamination by maternal DNA, the method comprising the steps of
obtaining, by a sequence analysis engine, sequencing data obtained from a sample comprising fetal DNA, and sequencing data obtained from a sample comprising maternal DNA;
comparing, by the sequence analysis engine, the sequencing data obtained from the sample comprising fetal DNA with the sequencing data obtained from the sample comprising maternal DNA to thereby detect variants;
calculating, by the sequence analysis engine, a difference in allele fractions using the variants of the fetal DNA and the variants of the maternal DNA; and
calculating, by the sequence analysis engine, purity using a distribution of difference in allele fractions.

18. The non-transient computer readable medium of claim 17, wherein the sample comprising fetal DNA comprises a fraction of whole blood.

19. The non-transient computer readable medium of any one of claims 17-18, wherein the sequencing data are whole genome sequencing data.

20. The non-transient computer readable medium of any one of claims 17-19, wherein the step of comparing comprises an incremental location-guided alignment.

21. The non-transient computer readable medium of any one of claims 17-20, wherein the step of calculating comprises identifying a peak value in the distribution of difference in allele fractions and multiplying the peak value by 2.

22. The non-transient computer readable medium of any one of claims 17-21, wherein the step of calculating the difference in allele fraction uses a step of determination of allele fractions AF
$${AF}_{M + D} = \frac{{M}_{B} ( 1 - α ) + {D}_{B} α}{( {M}_{A} + {M}_{B} ) ( 1 - α ) + ( {D}_{A} + {D}_{B} ) α}$$
wherein M_{A} and M_{B} or D_{A} and D_{B} are the copy numbers of the A and B alleles in the maternal (or daughter) sample, respectively, and wherein M_{A}+M_{B}=2 or D_{A} and D_{B}=2 for a diploid genome.

23. The non-transient computer readable medium of claim 22, wherein the step of calculating the difference in allele fraction is determined using
$$Δ AF = {AF}_{M + D} - {AF}_{M} = \frac{{M}_{B} ( 1 - α ) + {D}_{B} α}{( {M}_{A} + {M}_{B} ) ( 1 - α ) + ( {D}_{A} + D ) α} - \frac{{M}_{B}}{( {M}_{A} + {M}_{B} )} .$$

24. The non-transient computer readable medium of any one of claims 17-23, wherein the step of calculating the purity is determined using
$$α = \vert \frac{2 Δ AF}{{F}_{B} - {M}_{B}} \vert .$$
